(19) United States
(12) Patent Application Publication
(10) Pub. No.: US 2022/0088347 A1
(43) Pub. Date: Mar. 24, 2022

(54) SYSTEMS, APPARATUS, AND METHODS FOR STRESS AND ANXIETY MANAGEMENT, ACTIVE MEDITATION, AND REDIRECTING EMOTIONS

(71) Applicant: Jennifer Joffe, Portland, OR (US)

(72) Inventor: Jennifer Joffe, Portland, OR (US)

(21) Appl. No.: 17/479,327

(22) Filed: Sep. 20, 2021

Related U.S. Application Data
(60) Provisional application No. 63/080,446, filed on Sep. 18, 2020.

(51) Int. Cl. A61M 21/02; A61H 1/00

(52) U.S. Cl. CPC A61M 21/02; A61M 2021/0022; A61H 1/005

(57) ABSTRACT

A rotational apparatus comprising a platform and a base. The platform comprises a platform top, a platform bottom, and an edge, and the platform is configured to support a weight of a user. The base comprises a base top, a base bottom, and a rotatable element. The base top is coupled to and centered on the platform bottom. The rotatable element of the base is configured to rotate the platform in relation to the base. The rotational apparatus is configured for the platform to be parallel to the base while the platform rotates in relation to the base. The rotational apparatus is also configured to prevent rotation of the platform in relation to the base when the platform is tilted to contact a surface the base bottom is in contact with. A method of using the rotational apparatus while standing for stress or anxiety management in conjunction with rhythmic or repetitive movement. A method of using the rotational apparatus while sitting for stress or anxiety management in conjunction with rhythmic or repetitive movement.

### CROSS REFERENCE TO RELATED APPLICATIONS

This application claims priority to copending U.S. Application Ser. No. 63/080,446, filed on Sep. 18, 2020, which is hereby incorporated by reference for all purposes.

### BACKGROUND

The present disclosure relates generally to systems, apparatus, and methods for stress and anxiety management, active meditation, and redirecting emotions. In particular, systems, apparatus, and methods for stress and anxiety management, active meditation, and redirecting emotions using a rotational apparatus are described.

Humans are designed to move yet we live in a time where we sit more than ever. Cars, couches, offices, meetings, smart phones, and laptops have us with sore backs, our eyes glazed over and often we are left feeling stressed, drained and dissociated. When we are disconnected from our feelings we are disconnected from our bodies. Therapists have recognized that we only need to ride a 60 to 90 second wave of emotion, feeling it and processing it without making a decision that may harm us (think eating the thing, drinking the thing, sending the angry email) in order to decrease the odds that we will make a poor choice. By focusing on breathing, possibly repeating a mantra, while doing rhythmic and/or repetitive motions, an individual may achieve an overall calmer state of being, centeredness, and ability to be present throughout the day. Movement in conjunction with silence, once doing a rhythmic and/or repetitive motion is established, may allow the brain to release focus on the motion and be silent while the body remains in the motion.

Known systems, apparatus, and methods are not entirely satisfactory for the range of applications in which they are employed. For example, yoga generally focuses on meditation through static poses. While an individual moves from one to pose to another, the mind is engaged in the physical movement. It is not until the body remains still in the pose that the mind is meant to focus on the goal of meditation, relaxation, etc. As such, yoga is movement followed by silence which disrupts the ability of the brain to release focus on the movement.

Thus, there exists a need for systems, apparatus, and methods that improve upon and advance known systems, apparatus, and methods. Examples of new and useful systems, apparatus, and methods relevant to the needs existing in the field are discussed below.

### SUMMARY

The present disclosure is directed to a rotational apparatus comprising a platform and a base. The platform comprises a platform top, a platform bottom, and an edge, and the platform is configured to support a weight of a user. The base comprises a base top, a base bottom, and a rotatable element. The base top is coupled to and centered on the platform bottom. The rotatable element of the base is configured to rotate the platform in relation to the base. The rotational apparatus is configured for the platform to be parallel to the base while the platform rotates in relation to the base. The rotational apparatus is also configured to prevent rotation of the platform in relation to the base when the platform is tilted to contact a surface the base bottom is in contact with.

### BRIEF DESCRIPTION OF THE DRAWINGS

FIG. 1A is a perspective view of a first example of a rotational apparatus.

FIG. 1B is a bottom view of the rotational apparatus shown in FIG. 1A.

FIG. 1C is a perspective view of the bottom of the rotational apparatus shown in FIG. 1A.

FIG. 2A is a perspective view of a second example of a rotational apparatus.

FIG. 2B is a bottom view of the rotational apparatus shown in FIG. 2A.

FIG. 2C is a perspective view of the bottom of the rotational apparatus shown in FIG. 2A.

FIG. 3 illustrates a flow chart for using a rotational apparatus.

FIG. 4 illustrates a flow chart for stress and anxiety management, active meditation, and redirecting emotions.

FIG. 5 illustrates a flow chart for using a rotational apparatus while standing.

FIG. 6 illustrates a flow chart for using a rotational apparatus with one or two hands.

FIG. 7 illustrates a flow chart for using a rotational apparatus for physical exercise.

### DETAILED DESCRIPTION

The disclosed systems, apparatus, and methods will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, a variety of system, apparatus, and method examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire-based connector, whether directly or indirectly through a communication network.

“Controllably coupled” means that an electronic device controls operation of another electronic device.

Systems, Apparatus, and Methods for Stress and Anxiety Management, Active Meditation, and Redirecting Emotions

With reference to the figures, systems, apparatus, and methods for stress and anxiety management, active meditation, and redirecting emotions will now be described. The rotational apparatus discussed herein functions to build emotional, mental, and physical resilience and improve balance, coordination, posture and brain power, and emotional bandwidth in a user. When the user feels anxiety, stress, a flood of emotions, or a need for active meditation, the user engages with the rotational apparatus. Adults as well as children can benefit from engaging with the rotational apparatus. Adults and children on the autism spectrum may find particular benefits in assisting with managing stress and emotions. Additional benefits of engaging with the rotational apparatus include improvements in physical health, including digestive health.

The reader will appreciate from the figures and description below that the presently disclosed rotational apparatus and related methods address many of the shortcomings of conventional widgets. For example, yoga generally focuses on meditation through static poses. While an individual moves from one to pose to another, the mind is engaged in the physical movement. It is not until the body remains still in the pose that the mind is meant to focus on the goal of meditation, relaxation, etc. As such, yoga is movement followed by silence. The rotational apparatus provides a convenient, easy, an safe way to engage in a rhythmic and/or repetitive motion to free the mind to process emotions or meditate.

### Rotational Apparatus Embodiment One

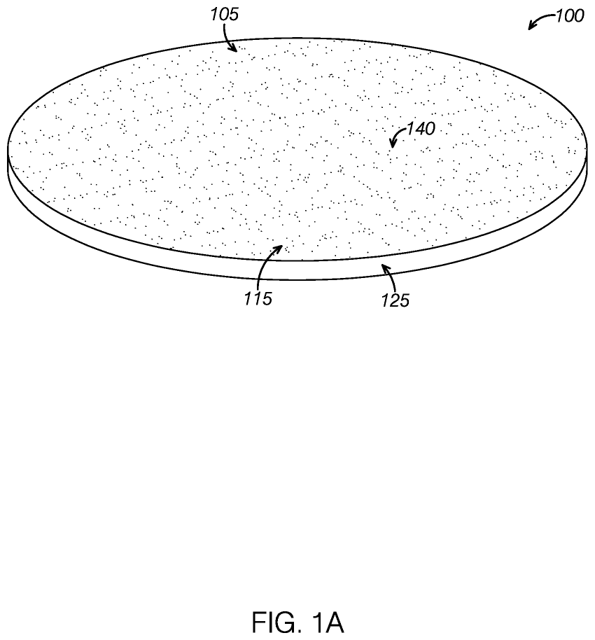

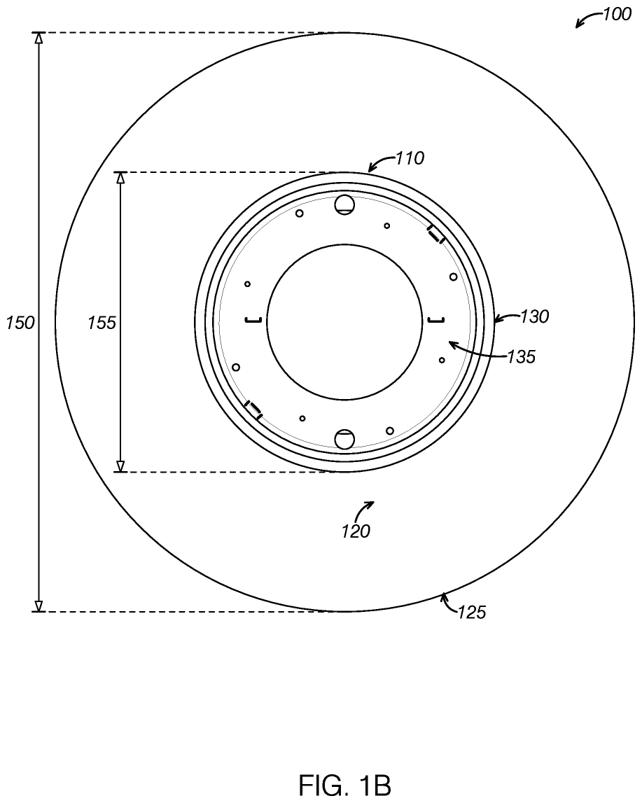

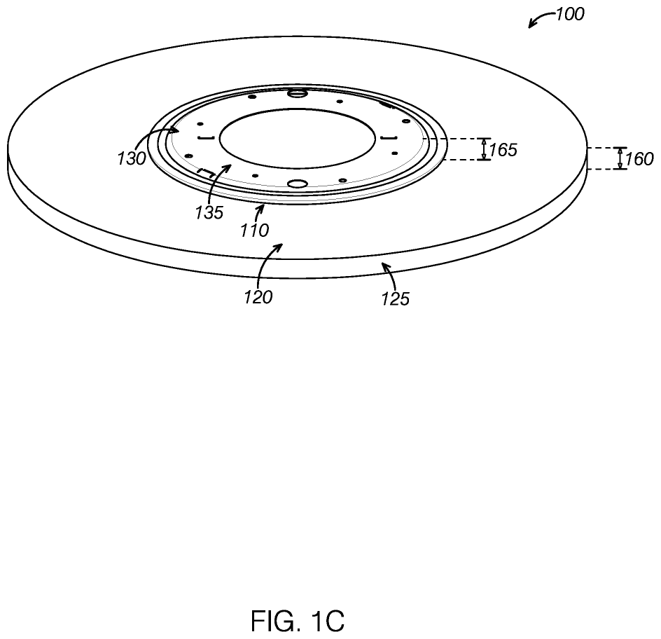

With reference to FIGS. 1A-1C, an example of a rotational apparatus 100 will now be described. The rotational apparatus 100 includes a platform 105 and a base 110, wherein the platform 105 includes a platform top 115, a platform bottom 120, edge 125, nonslip material 140, and the base 110 includes a rotational element 130, a base top (not shown) and a base bottom 135. The platform 105 has a first diameter 150 and the base 110 has a second diameter 155. The edge 125 has first thickness 160 and the base 110 has second thickness 165.

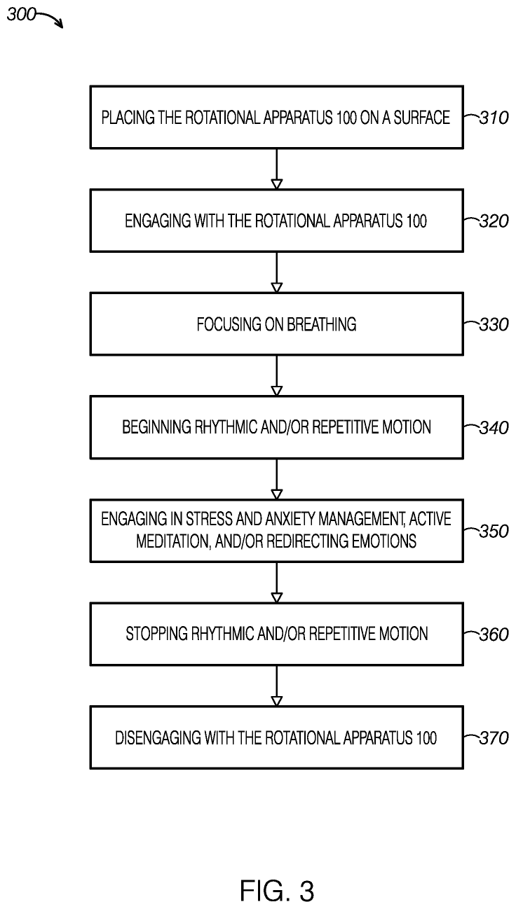

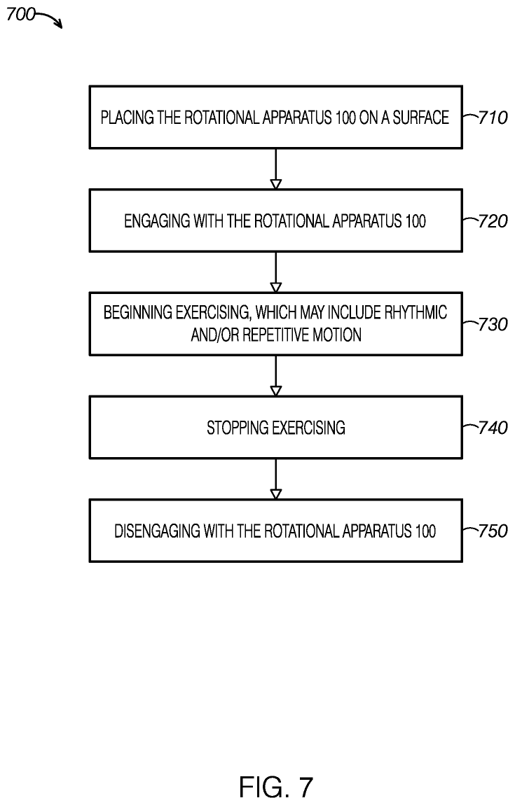

A user may engage with the rotational apparatus 100 for stress and anxiety management, active meditation, and/or redirecting emotions, as described in more detail below for the methods illustrated in FIGS. 3-6. The user may engage with the rotational apparatus 100 for physical activity or exercise, as described in more detail below for the method illustrated in FIG. 7.

To engage with the rotational apparatus 100, the user may place their full weight on the rotational apparatus 100. The rotational apparatus 100 can be robust enough to handle a weight of a large adult user. In one embodiment, the rotational apparatus 100 supports up to 400 pounds.

In one embodiment, the rotational apparatus 100 may be a portable device and a weight of the rotational apparatus 100 provides relative ease of portability for the user while remaining heavy enough to remain stable during use. In one embodiment, the weight of the rotational apparatus 100 is approximately ten pounds. The platform 105 may remain parallel to the base 110 while rotating. The user should not struggle to maintain their balance on the platform 105 while rotating.

In some examples, the rotational apparatus 100 does not include one or more features included in rotational apparatus 100. For example, some rotational apparatus examples do not include the nonslip material 140.

In other examples, the rotational apparatus 100 includes additional or alternative features, such as a second platform connected to the base bottom 135 to increase stability, a locking mechanism to prevent rotation, a nonslip material for the base bottom 135 to prevent slippage of the rotational apparatus 100. The rotational apparatus 100 may also include an attachment element configured to couple the rotational apparatus 100 to a vertical surface. Other additional or alternative features include one or more grips or handles.

Platform 105

As can be seen in FIGS. 1A-1C, the platform 110 includes the platform top 115, the platform bottom 120, the edge 125 and the nonslip material 140. The platform 105 provides a space for the user to physically engage with the rotational apparatus 100, such as by standing on, sitting on, kneeling on, or by using their hands to rotate the platform 105 or move the rotational apparatus 100. The nonslip material 140 prevents slippage of the user, such as the user's feet, while the user is engaged with the platform 105. Various types of material may be used for the nonslip material 140, such as rubber with small bumps on its surface. Other configurations of the nonslip material 140 may be used, such as the material the platform is comprised of having nonslip properties. FIG. 1A illustrates the nonslip material 140 covering the entire platform top 115. The nonslip material 140 may comprise various colors, textures, patterns, and designs.

The platform 105 may be made from a variety of materials, such as wood, plastic, composite materials, metal, etc. When the platform 105 is made of wood, various types may be used, such as Baltic birch. The platform 105 may also have a variety of shapes, such as round, oval, square, rectangular, octagonal, etc. As shown in FIGS. 1A-1C, the platform has a round shape with a slightly rounded edge 125.

The diameter, first diameter 150, of the platform 105 may be wide enough for an adult user to stand comfortably on the rotational apparatus 100 while maintaining their balance when rotating the platform 105 in relation to the base 110. In the example shown in FIGS. 1A-1C, the first diameter 150 is twenty-four (24) inches and the thickness of the platform 105, the first thickness 160, is three quarters of an inch (¾ inches).

The platform bottom 120 is coupled to the base top.

Base 110

In the example shown in FIGS. 1B and 1C, the base 110 includes the rotational element 130, the base top (not shown), and the base bottom 135. The rotational element 130 shown in FIGS. 1B and 1C is Triangle Manufacturing Company's part 12C: Steel Round Lazy Susan Turntable Bearing, 22 Gauge—12″. The specifications of this turntable are found on the Triangle Manufacturing Company's website: www.triangleoshkosh.com/lazy-susan-turntable-bearing-12c. Similar types of mechanisms may be used for the rotational element 130 or attached to, combined with, or incorporated into the base 110 that allow the platform 105 to rotate in relation to the base 110. In the example shown in FIGS. 1B and 1C, the rotational element 130 is the base 110, whereby the base top and the base bottom 135 of the base 110 is the top and the bottom of the rotational element 130.

As shown in FIGS. 1B and 1C, the base 110 is attached to the platform bottom 120. Four screws (not shown) secure this attachment. In other embodiments, a different number of screws may be used, or a different type of attachment may be used, such as bolts.

The rotational element 130 from Triangle Manufacturing Company described above allows the user to rotate the platform 105 in relation to the base 110 with minimal resistance. In another embodiment, the resistance provided by the base 110 when rotating the platform 105 is greater than the Triangle Manufacturing Company's product described above so that the user may use more force to rotate the platform 105. In another embodiment, the rotational element 130 and/or the base 110 provides multiple resistances to offer the user options to fit their use of the rotational apparatus 100.

The platform 105 remains parallel to the base 110 while rotating. The user should not struggle to maintain their balance on the platform 105 while rotating. When not being rotated, the platform 105 may tilt to contact a surface the rotational apparatus 100 is placed on while the base 110 is also still contacting the surface at or near the same location on the rotational apparatus 100 where the platform 105 is contacting the surface. For example, the user may use their foot to press down on the edge 125 the platform 105 until it contacts the surface to prevent rotation of the platform 105 in relation to the base 110. This may be done to make stepping on and off of the rotational apparatus 100 easier and safer for the user by preventing rotation while the user places their other foot onto the platform 105. Once the other foot is in the desired position on the platform 105, the user removes the foot from the edge 125.

The diameter, second diameter 155, of the base 110 is wide enough for an adult user to stand comfortably on the rotational apparatus 100 while maintaining their balance when rotating the platform 105 in relation to the base 110. In the example shown in FIGS. 1A-1C, the diameter of the base 110, the second diameter 155, is 12.063 inches, and the thickness of the base 110, the second thickness 165, is 0.313 inches. Other base diameters and thicknesses may be used while preserving the user's ability to maintain their balance when rotating the platform 105 in relation to the base 110.

The rotational apparatus 100 is configured to prevent rotation of the platform 105 in relation to the base 110 when the user tilts the platform 105 until it contacts a surface the base bottom is in contact with. The prevention of this rotation allows the user to engage with the rotational apparatus 100 slowly, easily, and safely. For example, when stepping onto or off of the platform top 105, it is advantageous for the platform 105 to not rotate in relation to the base 110 so that the user may steady themselves after placing one foot onto the platform top 115 before shifting their other foot onto the platform top 115. Methods for this type of use are described further in FIGS. 3-7.

In some examples the diameter of the platform 105, first diameter 150, is larger than the diameter of the base 110, second diameter 155 in order to provide this advantage of tilting the platform 105 to prevent rotation. In some examples, the first diameter 150 is at least 25% larger than the second diameter 155. The thicknesses of the platform 105 and the base 110 may also assist in providing the advantage of tilting of the platform 105 to prevent rotation. For example, the first and second thicknesses (160, 165) may be similar in dimension. It may be desirable for the second thickness 165 to not be too large, as this may increase the tilt of the platform 105 at a point which reduces the stability of the rotational apparatus 100 for the user. For example, if the second thickness 165 is too large, this may cause the tilt of the platform 105 to be too steep. Then when the user releases the pressure maintaining the tilt, the resulting movement of the platform 105 from the tilted position to a position where the platform 105 can be rotated in relation to the base 100 may be to abrupt and cause the user to lose their balance. In some examples, a second thickness 165 of six (6) inches may prevent adequate tilting of the platform 105. In one embodiment, the thickness of the platform, first thickness 160, is greater than or equal to the thickness of the base, second thickness 165.

### Additional Embodiments

With reference to the figures not yet discussed, the discussion will now focus on additional rotational apparatus embodiments. The additional embodiments include many similar or identical features to rotational apparatus 100. Thus, for the sake of brevity, each feature of the additional embodiments below will not be redundantly explained. Rather, key distinctions between the additional embodiments and rotational apparatus 100 will be described in detail and the reader should reference the discussion above for features substantially similar between the different rotational apparatus examples.

### Second Embodiment

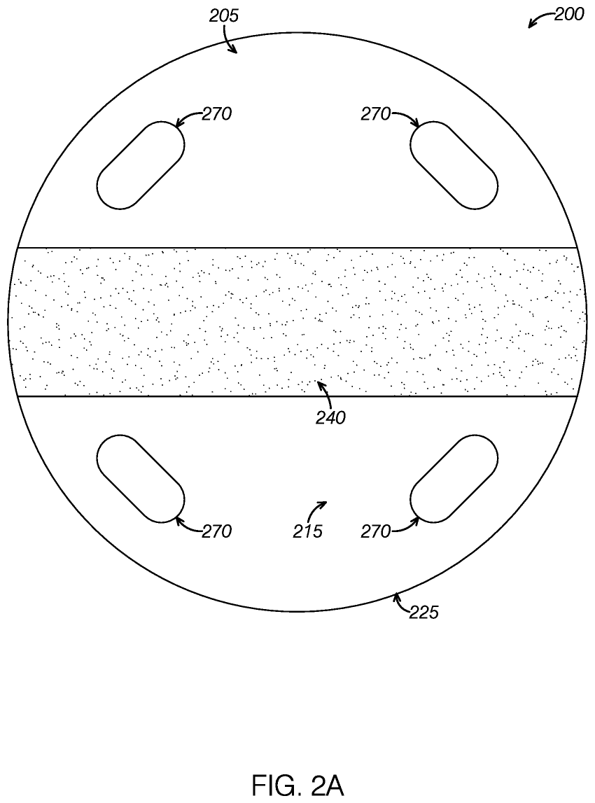

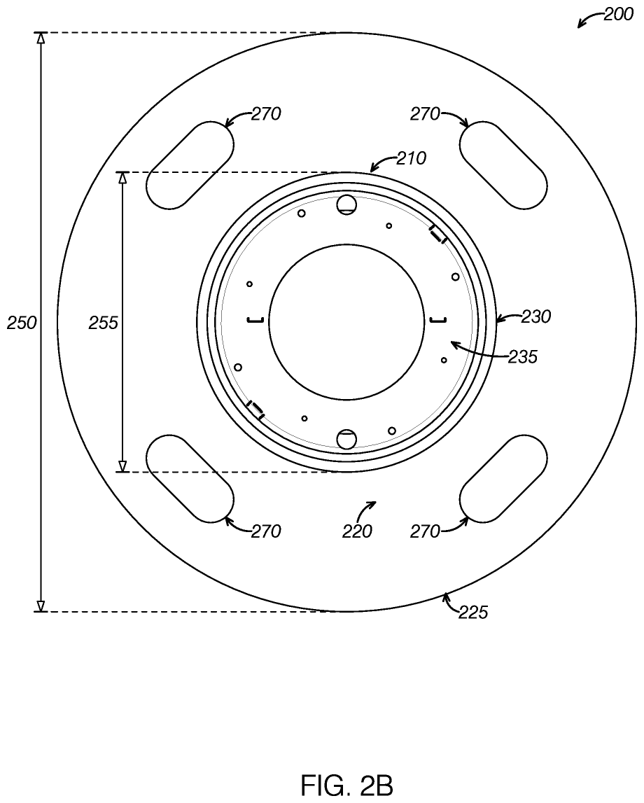

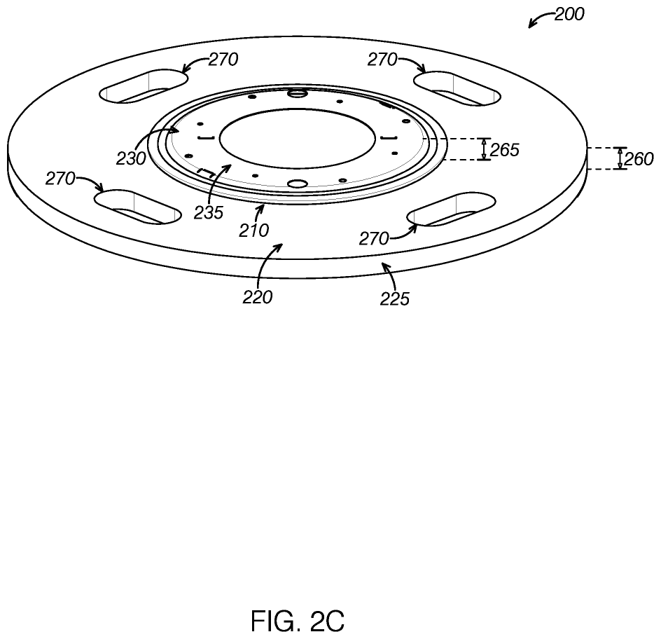

Turning attention to FIGS. 2A-2C, a second example of a rotational apparatus, rotational apparatus 200, will now be described. As can be seen in FIGS. 2A-2C, rotational apparatus 200 includes a platform 205 and a base 210, wherein the platform 205 includes a platform top 215, a platform bottom 220, edge 225, nonslip material 240, and one or more grips 270, and the base 210 includes a rotational element 230, a base top (not shown) and a base bottom 235. The platform 205 has a first diameter 250 and the base 210 has a second diameter 255. The edge 225 has first thickness 260 and the base 210 has second thickness 265.

The user may similarly engage with the rotational apparatus 200 as with rotational apparatus 100 for stress and anxiety management, active meditation, and/or redirecting emotions, as described in more detail below for the methods illustrated in FIGS. 3-6. The user may engage with the rotational apparatus 200 for physical activity, as described in more detail below for the method illustrated in FIG. 7.

In one embodiment, the rotational apparatus 200 may be a portable device aided by the use of the one or more grips 270. The weight of the rotational apparatus 200 provides relative ease of portability for the user while remaining heavy enough to remain stable during use. The platform 205 may remain parallel to the base 210 while rotating. The user should not struggle to maintain their balance on the platform 205 while rotating. In one embodiment, the weight of the rotational apparatus 200 is approximately ten pounds and the platform 205 made of wood.

Platform 205

As can be seen in FIGS. 2A-2C, the platform 205 includes the platform top 215, the platform bottom 220, the edge 225, the nonslip material 240, and the one or more grips 270. The platform 205 provides a space for a user to physically engage with the rotational apparatus 200, such as by standing on, sitting on, kneeling on, or by using their hands to rotate the platform 205 or move the rotational apparatus 200.

The nonslip material 240 prevents slippage of the user, such as the user's feet, while the user is engaged with the platform 205. Various types of material may be used for the nonslip material 240, such as rubber with small bumps on its surface. Other configurations of the nonslip material 140 may be used, such as the material the platform is comprised of having nonslip properties. FIG. 2A illustrates a rectangular shape of the nonslip material 240 attached to the platform top 215 in an area of the platform 205 where the user may stand, sit, or kneel. Other configurations of the nonslip material 240 may be used, such as two footprints located on the platform top 215 where the user may stand. The nonslip material 140 may comprise various colors, textures, patterns, and designs.

The one or more grips 270 provide a way for the user to hold onto the platform 205. FIGS. 2A-2C illustrate four grips 270 equidistant apart along the edge 225 of the platform 205. In other embodiments, the platform 205 may have less than or more than four grips 270. The one or more grips 270 may be on the platform top 215 or the platform bottom 220. As shown in FIGS. 2A-2C, the one or more grips 270 may extend through the platform 205. When two or more grips are present each grip is located equidistant from one another.

The platform 205 may be made from a variety of materials, such as wood, plastic, composite materials, metal, etc. When the platform 205 is made of wood, various types may be used, such as Baltic birch. The platform 205 may also have a variety of shapes, such as round, oval, square, rectangular, octagonal, etc. As shown in FIGS. 2A-2C, the platform 205 has a round shape with slightly rounded edge 225.

The diameter, first diameter 250, of the platform 205 may be wide enough for an adult user to stand comfortably on the rotational apparatus 200 while maintaining their balance while rotating the platform 105 in relation to the base 110.

The platform bottom 220 is coupled to the base top.

Base 210

In the example shown in FIGS. 2B and 2C, the base 210 includes the rotational element 230, the base top (not shown), and the base bottom 235. The rotational element 230 shown in FIGS. 2B and 2C is similar to rotational element 130. Similar types of mechanisms may be used for the rotational element 230 or attached to, combined with, or incorporated into the base 210 that allow the platform 205 to rotate in relation to the base 210. In the example shown in FIGS. 2B and 2C, the rotational element 230 is the base 210, whereby the base top and the base bottom 235 of the base 210 is the top and the bottom of the rotational element 230.

The rotational element allows the user to rotate the platform 205 in relation to the base 210 with minimal resistance. In another embodiment, the resistance provided by the base 210 when rotating the platform 205 is greater than the Triangle Manufacturing Company's product described above so that the user may use more force to rotate the platform 205. In another embodiment, the rotational element 230 and/or the base 210 provides multiple resistances to offer the user options to fit their use of the rotational apparatus 200.

The platform 205 remains parallel to the base 210 while rotating. The user should not struggle to maintain their balance on the platform 205 while rotating. When not being rotated, the platform 105 may tilt to contact a surface the rotational apparatus 200 is placed on while the base 210 is also still contacting the surface at or near the same location on the rotational apparatus 200 where the platform 205 is contacting the surface. For example, the user may use their foot to press down on the edge 225 the platform 205 until it contacts the surface to prevent rotation of the platform 205 in relation to the base 210. This may be done to make stepping on and off of the rotational apparatus 200 easier and safer for the user by preventing rotation while the user places their other foot onto the platform 205. Once the other foot is in the desired position on the platform 205, the user removes the foot from the edge 225.

The diameter, second diameter 255, of the base 210 is wide enough for an adult user to stand comfortably on the rotational apparatus 200 while maintaining their balance when rotating the platform 205 in relation to the base 210. Numerous base diameters and thicknesses may be used while preserving the user's ability to maintain their balance when rotating the platform 205 in relation to the base 210.

Just as rotational apparatus 100, the rotational apparatus 200 is configured to prevent rotation of the platform 205 in relation to the base 210 when the user tilts the platform 205 until it contacts a surface the base bottom is in contact with. The prevention of this rotation allows the user to engage with the rotational apparatus 200 slowly, easily, and safely. Methods for this type of use are described further in FIGS. 3-7.

In some examples the diameter of the platform 205, first diameter 250, is larger than the diameter of the base 210, second diameter 255 in order to provide this advantage of tilting the platform 205 to prevent rotation. The thicknesses of the platform 205 and the base 210 may also assist in providing the advantage of tilting of the platform 205 to prevent rotation. For example, the first and second thicknesses (260, 265) may be similar in dimension. It may be desirable for the second thickness 265 to not be too large, as this may increase the tilt of the platform 205 at a point which reduces the stability of the rotational apparatus 200 for the user.

Turning attention to FIG. 3, a method 300 of using the rotational apparatus 100 or 200 will now be described. Method 300 includes placing the rotational apparatus 100 on a surface at step 310, engaging with the rotational apparatus 100 at step 320, focusing on breathing at step 330, beginning rhythmic and/or repetitive motion at step 340, engaging in stress and anxiety management, active meditation, and/or redirecting emotions at step 350, stopping rhythmic and/or repetitive motion at step 360, and disengaging with the rotational apparatus 100 at step 370. The user may use the method 300 when feeling anxiety, stress, and/or a flooding of emotion, or a need for active meditation.

Step 310: placing the rotational apparatus 100 (or rotational apparatus 200) on a flat, stable surface. The location of the surface may be a location that is distraction-free or gives the user a sense of peace and/or calm. The location may include outdoor locations, such as a walkway, deck, or patio, or indoor locations such as a quiet room. For active meditation, the user may include candles, mood lighting, crystals, or other items. If the surface is soft, such as carpet or sand, a stabilizing device, such as a piece of wood, may be placed under the rotational apparatus 100 to provide additional support and stabilization.

The user engages with the rotational apparatus 100 at step 320. This may include the user stepping on to, sitting on, squatting on, or kneeling on the rotational apparatus 100, or the user being in close proximity to the rotational apparatus 100 in order to rotate the platform 105 with the user's hands or feet, or the user picking up the rotational apparatus 100. Part of engaging with the rotational apparatus 100 may be to prevent the rotation of the platform in relation to the base allowing the user to maintain their balance or get into a desired position. The user may also steady themselves by placing a hand or both hands on an object within reach of the rotation apparatus 100, such as a wall, doorjamb, heavy stool or chair, countertop, etc. before stepping on to, sitting on, squatting on, or kneeling on the rotational apparatus 100.

Once the user has engaged with the rotational apparatus 100, the user focuses on breathing in step 330. One example is BOX breathing utilizing a 4/4/4/4 technique. Another example has the user inhaling through their nose followed by exhaling for a longer duration than the inhale. In one embodiment, the user may also include speaking or thinking a mantra, such as before or during step 330.

In step 340, the user begins a rhythmic and/or repetitive movement. Basic movement may help the user's emotions move through the user's body and prevent them from getting stuck. In one embodiment, the rhythmic and/or repetitive movement is any motion that is twisting, spinning, or swaying the user's body. The user may also use arm movements, such as freely swinging, a jogging motion, a climbing motion, or a playful motion that brings the user comfort. These arm movements may be in addition to the twisting, spinning, or swaying of the user's body. While engaging in these movements, the user is simultaneously rotating the platform in relation to the base of the rotational apparatus 100.

In one embodiment, the user uses their hands to rotate the platform 105. The user may be kneeling next to the rotational apparatus 100, or may be seated next to the rotational apparatus 100, or may be lying prone next to the rotational apparatus 100, or may be seated or standing at a table or countertop with the rotational apparatus 100 on the table or countertop. Any position may be used which facilitates the user being able to rotate the platform 105 with their hand or hands. The user may use one hand or both hands to rotate the platform 105 and their hand or hands may be in any position on the platform, such as on the platform top 115 or the platform edge 125. If the rotatable platform 100 comprises one or more grips, the user may use the one or more grips to rotate the platform. The user may also benefit from the nonslip material 140 or surface to provide adequate grip for rotating the platform 100. The rhythmic and/or repetitive movement may be alternately rotating the platform 105 clockwise and counterclockwise with their hand or hands while twisting and lengthening their body to reach as far as possible in either direction.

In another embodiment, the user lays on their back and rotates the platform 105 with their feet. In one embodiment, the user stands on the rotational apparatus 100 and twists, stretches, and/or lengthens their body by placing their hands on a counter, chair or stool, doorway, or any heavy object to assist in the twisting, stretching, and/or lengthening and provide a deeper, more intense twist, stretch, and/or lengthening.

Once the user has achieved a desired rhythmic and/or repetitive movement, the user engages in stress and anxiety management, active meditation, and/or redirecting emotions in step 350. Any twisting, spinning, or swaying movements of the user's body may help move emotions through the body. When emotions are stirred up and moving within the user, adding physical motion may help move these emotions through the user so they do not get stuck.

In one embodiment, active meditation includes the user gently swinging their arms while rotating the rotational apparatus 100. In one embodiment, stress and anxiety management includes the user moving at a more rapid pace, such as moving their arms in a jogging motion. In one embodiment, redirecting emotions includes the user tapping their left shoulder with their right hand followed by their right shoulder with their left hand as they move. Any twisting, spinning, or swaying movements of the user's body may help move emotions through the body.

In one embodiment, the user processes troubled feelings, thoughts, or emotions while engaging in the rhythmic and/or repetitive movement until the feelings, thoughts, or emotions have decreased or past. In one embodiment, the rhythmic and/or repetitive movement provides stress relief for the user's body as the user's body and its systems practice working together without outside distractions. This may also benefit the user's cardiovascular, digestive, and immune systems by helping protect the user's body from harmful effects of stress.

When the user is ready to stop stress and anxiety management, active meditation, and/or redirecting emotions, the user stops the rhythmic and/or repetitive movement in step 360.

The user then disengages with the rotational apparatus 100 at step 370. This may include the user removing themselves from the top of the platform, such as stepping off of the platform, or by removing their hands from the platform, or by setting down the rotational apparatus 100. As in step 320, the user may steady themselves by placing a hand or both hands on an object within reach of the rotation apparatus 100, such as a wall, doorjamb, heavy stool or chair, countertop, etc. before stepping off of or removing themselves from the rotational apparatus 100.

An optional breathing step may be performed before or after step 370. The user takes one deep breath, inhaling through their nose, creating the sensation of filling their entire body with air, such as a deep belly breath, and then exhaling through their mouth. Other types of breathing or completion steps may be used. In one example for active meditation, a gratitude practice may be performed where the user says three things they are grateful for. In one example for stress and anxiety management, the user may set intentions for their day moving forward.

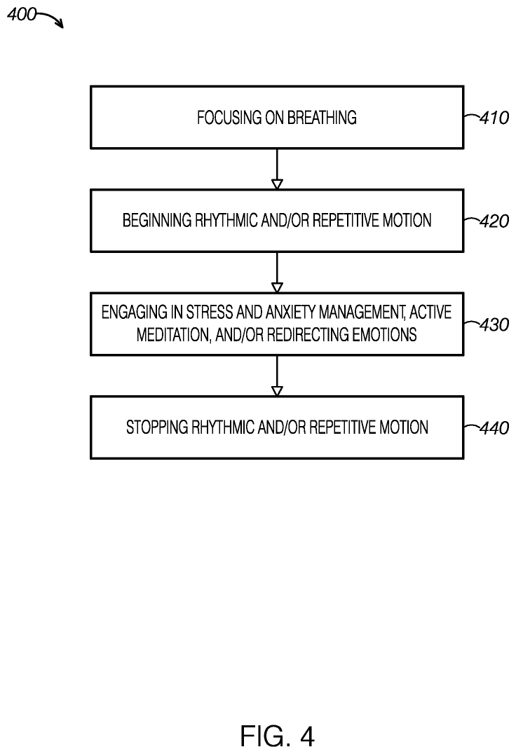

Turning attention to FIG. 4, an example of a method 400 for stress and anxiety management, active meditation, and/or redirecting emotions will now be described. Method 400 includes focusing on breathing at step 410, beginning rhythmic and/or repetitive motion at step 420, engaging in stress and anxiety management, active meditation, and/or redirecting emotions at step 430, and stopping rhythmic and/or repetitive motion at step 440. The user may initiate method 400 when feeling anxiety, stress, and/or a flooding of emotion, or a need for active meditation.

Before the user begins step 410, the user may select a location to begin that is distraction-free or gives them a sense of peace and/or calm. The locations may include outdoor locations, such as a walkway, deck, or patio, or indoor locations such as quiet room. For active meditation, the user may include candles, mood lighting, crystals, or other items.

The user focuses on breathing in step 410. One example is BOX breathing utilizing a 4/4/4/4 technique. Another example has the user inhaling through their nose followed by exhaling for a longer duration than the inhale. In one embodiment, the user may also include speaking or thinking a mantra either before or during step 410. In another embodiment, the mantra is spoken or thought throughout method 400.

In step 420, the user begins a rhythmic and/or repetitive movement. Basic movement may help the user's emotions move through the user's body and prevent them from getting stuck. In one embodiment, the rhythmic and/or repetitive movement is any motion that is twisting, spinning, or swaying. The user may also use arm movements, such as freely swinging, a jogging motion, a climbing motion, or in a playful motion that brings the user comfort. In one embodiment, the rhythmic and/or repetitive movement includes bilateral stimulation where the user engages their torso in a twisting motion coupled with the user's right arm making contact with the user's left side of the body repeated by the user's left arm making contact with the user's right side of the body with a simple touch or tap, while also allowing the user's eyes to move from one side of the location to the other, following the user's body. In one embodiment, an apparatus may be used to achieve the rhythmic and/or repetitive movement, such as rotational apparatus 100 or 200.

Once the user has achieved a desired rhythmic and/or repetitive movement, the user engages in in stress and anxiety management, active meditation, and/or redirecting emotions in step 430. In one embodiment, active meditation includes the user gently swinging their arms. In one embodiment, stress management includes the user moving at a more rapid pace, such as moving their arms in a jogging motion. In one embodiment, redirecting emotions includes the user tapping their left shoulder with their right hand followed by their right shoulder with their left hand as they move.

In one embodiment, the user processes troubled feelings or thoughts while engaging in the rhythmic and/or repetitive movement until the feelings have decreased or past. In one embodiment, the rhythmic and/or repetitive movement provides stress relief for the user's body as the user's body and its systems practice working together without outside distractions. This may also benefit the user's cardiovascular, digestive, and immune systems by helping protect the user's body from harmful effects of stress. In one embodiment, concentrating on the user's body movements may enhance the mind-body connection.

Eye Movement Desensitization and Reprocessing (EMDR) therapy is an extensively researched, effective psychotherapy method proven to help people recover from trauma and other distressing life experiences, including PTSD, anxiety, depression, and panic disorders. In one embodiment, the user may replicate EMDR like therapies where the user can sit with a troubling thought or memory, then engage in rotating the rotational apparatus creating a space for rapid eye movement as the user tracks a fixed spot in their line of vision. The user may pair this type of simulation with a powerful thought like “I survived it and I am strong.” This therapeutic process, the user's thoughts, feelings and behavior paired with the rapid eye movement and the rhythmic and/or repetitive movement may result in better emotional health, and overall wellbeing.

When the user is ready to stop stress and anxiety management, active meditation, and/or redirecting emotions, the user stops the rhythmic and/or repetitive movement in step 440.

An optional breathing step may be performed after step 440. The user takes one deep breath, inhaling through their nose, creating the sensation of filling their entire body with air, such as a deep belly breath, and then exhaling through their mouth. Other types of breathing or completion steps may be used. In one example for active meditation, a gratitude practice may be performed where the user says three things they are grateful for. In one example for stress management or stress, the user may set intentions for their day moving forward.

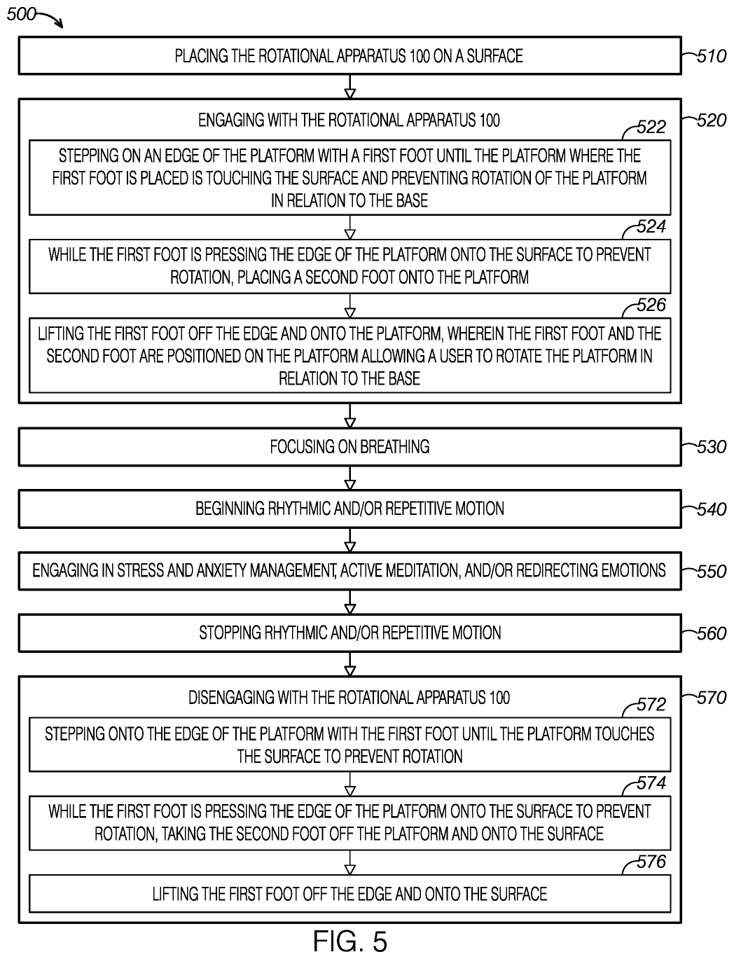

Turning attention to FIG. 5, a method 500 of using the rotational apparatus 100 (or rotational apparatus 200) while the user is standing on the platform 105 will now be described. Method 500 includes placing the rotational apparatus 100 on a surface at step 510, engaging with the rotational apparatus 100 at step 520, focusing on breathing at step 530, beginning rhythmic and/or repetitive motion at step 540, engaging in stress and anxiety management, active meditation, and/or redirecting emotions at step 550, stopping rhythmic and/or repetitive motion at step 560, and disengaging with the rotational apparatus 100 at step 570. The user may initiate method 500 when feeling anxiety, stress, and/or a flooding of emotion, or a need for active meditation.

Step 510: the user places the rotational apparatus 100 on a flat, stable surface. The location of the surface may be a location that is distraction-free or gives the user a sense of peace and/or calm. The location may include outdoor locations, such as a walkway, deck, or patio, or indoor locations such as a quiet room. For active meditation, the user may include candles, mood lighting, crystals, or other items. If the surface is soft, such as carpet or sand, a stabilizing device, such as a piece of wood, may be placed under the rotational apparatus 100 to provide additional support and/or stabilization.

The user engages with the rotational apparatus 100 at step 520 by stepping onto the platform top 115. At any time, the user may steady themselves by placing a hand or both hands on an object within reach of the rotation apparatus 100, such as a wall, doorjamb, heavy stool or chair, countertop, etc. In one embodiment, the user places a first foot fully onto the platform top 115. The placement of the first foot may be in a position where the user will ultimately have the first foot placed for rotation of the platform 105. The user then shifts their weight onto the first foot. The user lifts a second foot and places it fully onto the platform 105. The placement of the second foot may be in a position where the user will ultimately have the second foot placed for rotation of the platform 105.

In one embodiment, the user places the first foot onto the platform 105, near the edge 125, and presses the first foot downward until the edge 125 of the platform 105 underneath the first foot contacts the surface. This prevents the platform from rotating allowing the user to maintain their balance while stepping onto the platform 105. The user shifts their weight onto the first foot and, while maintaining the contact between the edge 125 of the platform 105 and the surface, the user places the second foot fully onto the platform top 115, such as in a position where the user will ultimately have the second foot placed for rotation of the platform 105. Next, the user shifts their weight from the first foot to the second foot. Then the user lifts the first foot and places it fully onto the platform top 115 while releasing the contact of the platform 105 with the surface. If not already in position, the user may then slowly and gradually shift the positions of their feet on the platform 105 to a comfortable and stable position for the user, such as shoulder width apart.

Once the user has engaged with the rotational apparatus 100, the user focuses on breathing in step 530. One example is BOX breathing utilizing a 4/4/4/4 technique. Another example has the user inhaling through their nose followed by exhaling for a longer duration than the inhale. In one embodiment, the user may also include speaking or thinking a mantra, such as before or during step 530.

In step 540, the user begins a rhythmic and/or repetitive movement. Basic movement may help the user's emotions move through the user's body and prevent them from getting stuck. In one embodiment, the rhythmic and/or repetitive movement is any motion that is twisting, spinning, or swaying while the user rotates the platform 105. The user may also use arm movements, such as freely swinging, a jogging motion, a climbing motion, or in a playful motion that brings the user comfort. In one embodiment, the rhythmic and/or repetitive movement includes bilateral stimulation where the user rotates on the platform 105 by engaging their torso in a twisting motion coupled with the user's right arm making contact with the user's left side of the body repeated by the user's left arm making contact with the user's right side of the body with a simple touch or tap, while also allowing the user's eyes to move from one side of the location to the other, following the user's body.

Once the user has achieved a desired rhythmic and/or repetitive movement, the user engages in in stress and anxiety management, active meditation, and/or redirecting emotions in step 550. In one embodiment, active meditation includes the user gently swinging their arms. In one embodiment, stress management includes the user moving at a more rapid pace, such as moving their arms in a jogging motion. In one embodiment, redirecting emotions includes the user tapping their left shoulder with their right hand followed by their right shoulder with their left hand as they move.

In one embodiment, the user processes troubled feelings or thoughts while engaging in the rhythmic and/or repetitive movement until the feelings have decreased or past. In one embodiment, the rhythmic and/or repetitive movement provides stress relief for the user's body as the user's body and its systems practice working together without outside distractions. This may also benefit the user's cardiovascular, digestive, and immune systems by helping protect the user's body from harmful effects of stress. In one embodiment, concentrating on the user's body movements may enhance the mind-body connection.

When the user is ready to stop stress and anxiety management, active meditation, and/or redirecting emotions, the user stops the rhythmic and/or repetitive movement in step 560.

The user then disengages with the rotational apparatus 100 at step 570. In one embodiment, the user shifts their weight to a first foot, then moves a second foot backward to the edge 125 of the platform 105 in a similar manner as step 520. The user steps on the edge 125 with the first foot until the platform 105 where the first food is placed is contacting the surface and preventing rotation of the platform 105 in relation to the base 110. The user the shifts their weight to the second foot and lifts and places the first foot onto the surface. The user then shifts their weight to the first foot and lifts the second foot off the edge 125 and onto the surface. As in step 520, the user may also steady themselves by placing a hand or both hands on an object within reach of the rotation apparatus 100, such as a wall, doorjamb, heavy stool or chair, countertop, etc.

An optional breathing step may be performed before or after step 570. The user takes one deep breath, inhaling through their nose, creating the sensation of filling their entire body with air, such as a deep belly breath, and then exhaling through their mouth. Other types of breathing or completion steps may be used. In one example for active meditation, a gratitude practice may be performed where the user says three things they are grateful for. In one example for stress and anxiety management, the user may set intentions for their day moving forward.

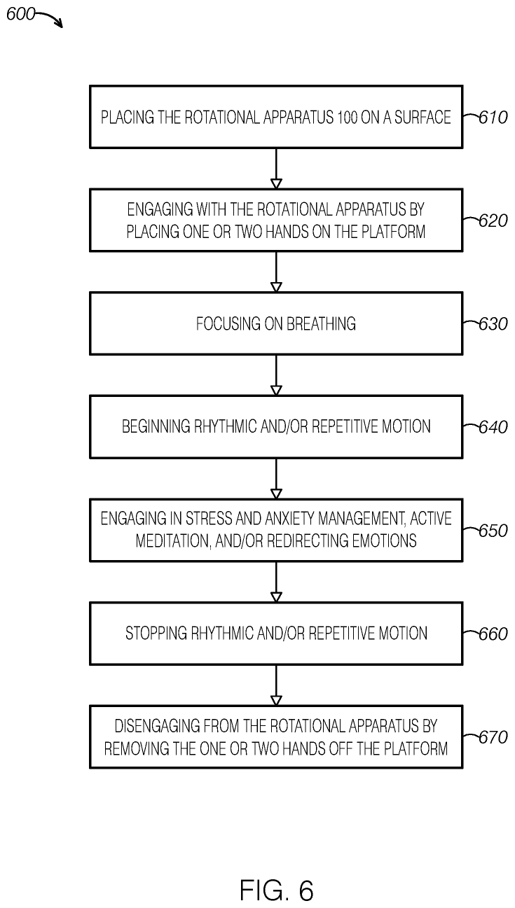

Turning attention to FIG. 6, a method 600 of using the rotational apparatus 100 (or rotational apparatus 200) with one or two hands will now be described. Method 600 includes placing the rotational apparatus 100 on a surface at step 610, engaging with the rotational apparatus 100 at step 620, focusing on breathing at step 630, beginning rhythmic and/or repetitive motion at step 640, engaging in stress and anxiety management, active meditation, and/or redirecting emotions at step 650, stopping rhythmic and/or repetitive motion at step 660, and disengaging with the rotational apparatus 100 at step 670. The user may initiate method 600 when feeling anxiety, stress, and/or a flooding of emotion, or a need for active meditation.

Step 610: the user places the rotational apparatus 100 on a flat, stable surface. The surface may be the floor of a structure, such as a therapist's office or a room within a home. The surface may be an elevated surface such as a table or countertop. The location of the surface may be a location that is distraction-free or gives the user a sense of peace and/or calm. The location may include outdoor locations, such as a walkway, deck, or patio, or indoor locations such as a quiet room. For active meditation, the user may include candles, mood lighting, crystals, or other items. If the surface is soft, such as carpet or sand, a stabilizing device, such as a piece of wood, may be placed under the rotational apparatus 100 to provide additional support and/or stabilization.

The user engages with the rotational apparatus 100 at step 620. In one embodiment, the user begins by placing one or two hands on the platform top 115 or the platform edge 125. In one example, the user places their right hand on a right side of the platform top 115 and places their left hand on a left side of the platform top 115. The left and right hands may be equidistance from the platform edge 125. If the rotatable platform 100 comprises one or more grips, the user may use the one or more grips to rotate the platform. The user may also benefit from the nonslip material 140 or surface to provide adequate grip for rotating the platform 100.

In an optional step, the user places themselves in a position to comfortably place their hand or hands on the platform 105. The user may be kneeling next to the rotational apparatus 100, or may be seated next to the rotational apparatus 100, or may be lying prone next to the rotational apparatus 100, or may be seated or standing at a table or countertop with the rotational apparatus 100 on the table or countertop. Any position may be used which facilitates the user being able to rotate the platform 105 with their hand or hands.

Once the user has engaged with the rotational apparatus 100, the user focuses on breathing in step 630. One example is BOX breathing utilizing a 4/4/4/4 technique. Another example has the user inhaling through their nose followed by exhaling for a longer duration than the inhale. In one embodiment, the user may also include speaking or thinking a mantra, such as before or during step 630.

In step 640, the user begins a rhythmic and/or repetitive movement. Basic movement may help the user's emotions move through the user's body and prevent them from getting stuck. In one embodiment, the rhythmic and/or repetitive may be alternately rotating the platform 105 clockwise and counterclockwise with their hand or hands while twisting and lengthening their body to reach as far as possible in either direction.

Once the user has achieved a desired rhythmic and/or repetitive movement, the user engages in in stress and anxiety management, active meditation, and/or redirecting emotions in step 650. In one embodiment, active meditation includes the user gently swinging their arms. In one embodiment, stress management includes the user moving at a more rapid pace, such as moving their arms in a jogging motion. In one embodiment, redirecting emotions includes the user tapping their left shoulder with their right hand followed by their right shoulder with their left hand as they move.

In one embodiment, the user processes troubled feelings or thoughts while engaging in the rhythmic and/or repetitive movement until the feelings have decreased or past. In one embodiment, the rhythmic and/or repetitive movement provides stress relief for the user's body as the user's body and its systems practice working together without outside distractions. This may also benefit the user's cardiovascular, digestive, and immune systems by helping protect the user's body from harmful effects of stress. In one embodiment, concentrating on the user's body movements may enhance the mind-body connection.

When the user is ready to stop stress and anxiety management, active meditation, and/or redirecting emotions, the user stops the rhythmic and/or repetitive movement in step 660.

The user then disengages with the rotational apparatus 100 at step 670. In one embodiment, the user presses on the edge 125 of the platform 105 with their hand until the platform 105 where the hand is placed is contacting the surface. This prevents the platform 105 from rotating in relation to the base 110. While the hand is preventing rotation of the platform 105, the user safely moves off of the platform 105 to the surface. Once comfortably and safely on the surface, the user lifts the hand off the edge 125 of the platform 105.

An optional breathing step may be performed before or after step 670. The user takes one deep breath, inhaling through their nose, creating the sensation of filling their entire body with air, such as a deep belly breath, and then exhaling through their mouth. Other types of breathing or completion steps may be used. In one example for active meditation, a gratitude practice may be performed where the user says three things they are grateful for. In one example for stress and anxiety management, the user may set intentions for their day moving forward.

Turning attention to FIG. 7, an example of a method 700 of using the rotational apparatus 100 (or rotational apparatus 200) for physical exercise will now be described. Method 700 includes placing the rotational apparatus 100 on a surface at step 710, engaging with the rotational apparatus 100 at step 720, beginning exercising, which may include rhythmic and/or repetitive motion at step 730, stopping exercising at step 740, and disengaging with the rotational apparatus 100 at step 750. The user may initiate the method 700 to improve physical conditioning, health, and/or recreational or professional sporting skills.

Step 710: the user places the rotational apparatus 100 on a flat, stable surface. The location of the surface may include outdoor locations, such as a walkway, deck, or patio, or indoor locations such as quiet room. If the surface is soft, such as carpet or sand, a stabilizing device, such as a piece of wood, may be placed under the rotational apparatus 100 to provide additional support and stabilization.

The user engages with the rotational apparatus 100 at step 720. This may include the user stepping on to, sitting on, squatting on, or kneeling on the rotational apparatus 100, or the user being in close proximity to the rotational apparatus 100 in order to rotate the platform 105 with the user's hands or feet, or the user picking up the rotational apparatus 100.

In step 730, the user begins exercising, which may include a rhythmic and/or repetitive movement. In one embodiment, the rhythmic and/or repetitive movement is any motion that is twisting, spinning, or swaying the user's body while rotating the platform 105. The user may also use arm movements, such as freely swinging, a jogging motion, a climbing motion, or in a playful motion. The arm movements may be in addition to the twisting, spinning, or swaying of the user's body. In one embodiment, the user is kneeling next to the rotational apparatus 100 and using their hands to rotate the platform 105. In one example, the rotational apparatus 100 is placed on a table or countertop for the user to use their hands to rotate the platform 105. The rhythmic and/or repetitive movement may be alternately rotating the platform 105 clockwise and counterclockwise with their hands while twisting and lengthening their body to reach as far as possible in each direction. In one embodiment, the user lays on their back and rotates the platform 105 with their feet. In one embodiment, the user stands on the rotational apparatus 100 and twists, stretches, and/or lengthens their body by using their hands on a counter, chair or stool, doorway, or any heavy object to assist in the twisting, stretching, and/or lengthening and provide a deeper, more intense twist, stretch, and/or lengthening.

In one embodiment, the user stands on the platform 105 and twists their body while moving their arms and hands side to side, mimicking a boxing motion. In one embodiment, the user stands on the platform 105 and moves their body in a golf swing motion. In one embodiment, the user stands on the platform 105 and uses lateral motions with leg strength. This may include motions to improve skills for sports, such as downhill skiing, waterskiing, wake boarding, skateboarding, etc. The user stands on the platform 105 with their legs together, bends their knees and uses their upper body to create resistance and a motion that replicates a hopping-like motion working the core abdominal muscles which act as stabilizers, the gluteus maximus (in the buttocks and thighs) which supports legs movements, and your quadriceps which support extending.

In step 740, the user stops exercising. In one embodiment, the user slowly reduces the frequency and speed of the rhythmic and/or repetitive movements.

The user then disengages with the rotational apparatus 100 at step 750. This may include the user removing themselves from the platform top 115, such as stepping off of the platform 105, or by removing their hands from the platform 105, or by setting down the rotational apparatus 105.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A rotational apparatus, comprising:
a platform
comprising a platform top, a platform bottom, and an edge, and
configured to support a weight of a user;
a base
comprising a base top, a base bottom, and a rotatable element,
wherein the base top is coupled to and centered on the platform bottom, and
wherein the rotatable element is configured to rotate the platform in relation to the base; and
wherein the rotational apparatus is configured for the platform to be parallel to the base while the platform rotates in relation to the base, and
wherein the rotational apparatus is configured to prevent rotation of the platform in relation to the base when the platform is tilted to contact a surface the base bottom is in contact with.

2. The rotational apparatus of claim 1, further comprising one or more grips located near the edge, wherein when two or more grips are present each grip is located equidistant from one another.

3. The rotational apparatus of claim 2, wherein the one or more grips extend through the platform.

4. The rotational apparatus of claim 2, wherein the one or more grips are indentations in the platform to fit a hand or foot of the user.

5. The rotational apparatus of claim 1, wherein the rotational element is a lazy susan turntable.

6. The rotational apparatus of claim 1, further comprising a nonslip material coupled to the platform top.

7. The rotational apparatus of claim 7, wherein the nonslip material covers all of the platform top.

8. The rotational apparatus of claim 7, wherein the nonslip material covers a portion of the platform top.

9. The rotational apparatus of claim 1, wherein the platform is comprised of a material with nonslip properties.

10. A method of using a rotational apparatus, comprising:
placing the rotational apparatus on a flat, stable surface,
wherein the rotational apparatus comprises a platform comprising a platform top, a platform bottom, and an edge and configured to support a weight of a user and a base comprising a rotational element configured to rotate the platform in relation to the base, wherein the base is centered on and coupled to the platform bottom,
wherein the rotational apparatus is configured for the platform to be parallel to the base while the platform rotates in relation to the base, and
wherein the rotational apparatus is configured to prevent rotation of the platform in relation to the base when the platform is tilted to contact the surface;
engaging with the rotational apparatus by
stepping on the edge with a first foot until the platform where the first foot is placed is contacting the surface and preventing rotation of the platform in relation to the base,
while the first foot is preventing rotation of the platform in relation to the base, placing a second foot onto the platform top, and
lifting the first foot off the edge and onto the platform top such that the first foot and the second foot are positioned on the platform top allowing the user to rotate the platform in relation to the base;
focusing on breathing;
beginning a rhythmic or repetitive movement while rotating the platform in relation to the base;
engaging in stress or anxiety management in conjunction with the rhythmic or repetitive movement;
stopping the rhythmic or repetitive movement; and
disengaging from the rotational apparatus by
stepping on the edge with the first foot until the platform where the first food is placed is contacting the surface and preventing rotation of the platform in relation to the base,
while the first foot is preventing rotation of the platform in relation to the base, lifting the second foot off the platform top and onto the surface, and
lifting the first foot off the edge and onto the surface.

11. The method of claim 10, further comprising speaking or thinking a mantra.

12. The method of claim 10, wherein the rhythmic or repetitive movement comprises one or more of a twisting, spinning, or swaying motion.

13. The method of claim 10, wherein the rhythmic or repetitive movement comprises arm movements comprising one or more of freely swing, a jogging motion, a climbing motion, or a playful motion, wherein the arm movements may be in addition to a twisting, spinning, or swaying motion.

14. The method of claim 10, wherein engaging in stress and anxiety management comprises one or more of active meditation or redirecting emotions.

15. The method of claim 10, wherein engaging in stress and anxiety management comprises one or more of processing troubled feelings or thoughts.

16. The method of claim 10, further comprising focusing on breathing after stopping the rhythmic or repetitive movement.

17. A method of using a rotational apparatus, comprising:
placing the rotational apparatus on a flat, stable surface,
wherein the rotational apparatus comprises a platform comprising a platform top, a platform bottom, and an edge and configured to support a weight of a user and a base comprising a rotational element configured to rotate the platform in relation to the base, wherein the base is centered on and coupled to the platform bottom,
wherein the rotational apparatus is configured for the platform to be parallel to the base while the platform rotates in relation to the base, and
wherein the rotational apparatus is configured to prevent rotation of the platform in relation to the base when the platform is tilted to contact the surface;
engaging with the rotational apparatus by placing one or two hands on the platform;
focusing on breathing;
beginning a rhythmic or repetitive movement while rotating the platform in relation to the base;
engaging in stress or anxiety management in conjunction with the rhythmic or repetitive movement;
stopping the rhythmic or repetitive movement; and
disengaging from the rotational apparatus by removing the one or two hands off the platform.

18. The method of claim 18, wherein the rhythmic or repetitive movement comprises alternately rotating the platform clockwise and counterclockwise while twisting and lengthening the user's body to reach as far as possible in either direction.

19. The method of claim 18, wherein engaging in stress and anxiety management comprises one or more of active meditation, redirecting emotions, or processing troubled feelings or thoughts.

20. The method of claim 18, wherein engaging with the rotational apparatus further comprises the user laying, sitting, kneeling, or standing before the rotational apparatus before placing the one or two hands on the platform top or the platform edge.
